(19) United States
(12) Patent Application Publication
(10) Pub. No.: US 2005/0048567 A1
(43) Pub. Date: Mar. 3, 2005

(54) Internal standards for sphingolipids

(76) Inventors: Bryan Winchester, London (GB); Kevin Mills, London (GB)

Correspondence Address: Leon R. Yankwich;Yankwich & Associates, 201 Broadway, Cambridge, MA, 02139, US

(21) Appl. No.: 10/497,218

(22) PCT Filed: Nov. 27, 2002
(86) PCT No.: PCT/GB02/05327

(30) Foreign Application Priority Data
Nov. 28, 2001 (GB) 0128498.3

(51) Int. Cl. G01N 33/53; G01N 33/20

(52) U.S. Cl. 435/7.1; 436/84

(57) ABSTRACT

The present invention relates to an internal standard for use in mass spectometry to determine the concentration of a test sphingolipid, the internal standard comprising the same oligosaccharyl chain and long chain base sphingosine as the test sphingolipid and having an acyl group of different mass from the test sphingolipid but providing a sphingolipid of the same chemical nature as the test sphingolipid, and methods for the preparation and use thereof.

The present invention relates to internal standards for sphingolipids to allow measurement of sphingolipids by mass spectrometry and provide for diagnosis of lysosomal storage disorders.

Sphingolipid is a generic name for lipids having a long chain base sphingoid such as glycosphingolipids, sphingophospholipids and ceramides. As a common structure, sphingolipids have a ceramide structure in which a long chain fatty acid with non-uniform chain length is bonded to the amino group of the sphingoid via an acid-amide bond.

Lysosomal storage disorders are a large family of genetic disorders that can lead to the manifestation of severe clinical symptoms. Over forty lysosomal storage disorders are known, including Tay Sachs disease, Sandhoff disease, Fabry disease, Gaucher disease, Krabbe disease and Niemann Pick disease, and although the frequency of each individual lysosomal storage disease is relatively rare, the collective incidence of all lysosomal storage disorders is about 1 in 7000 newborns.

Each lysosomal storage disorder results from a deficiency in a lysosomal enzyme, transporter or protein involved in lysosomal biogenesis or function. The deficiency leads to the accumulation of storage products normally degraded in the lysosome. These storage products are characteristic of each defect and their accumulation is the initial step in the process leading to a lysosomal storage disease. As well as accumulating in cells within tissues, the storage products appear in body fluids such as plasma, cerebrospinal fluid and urine. The concentration of the storage products in blood or urine is a good marker of the progress of the disease or in the few diseases where treatment is possible of the progress of the treatment.

Accurate measurement of the concentration of specific storage products is therefore of great value in the diagnosis and monitoring of patients.

The glycosphingolipidises constitute the largest group of lysosomal storage diseases. One such disease is Fabry disease, an X-linked lysosomal storage disease resulting from a deficiency of the lysosomal hydrolase, α-galactosidase A (EC.3.2.1.22), which leads to the progressive accumulation within lysosomes of glycosphingolipids with terminal α-galactosyl residues.

The major storage product involved in Fabrys disease is ceramide trihexoside (CTH) or globotriaosylceramide (GbOse₃Cer, Gb₃, GL-3). Storage occurs predominantly in the endothelial, perithelial and smooth muscle cells of blood vessels but there is deposition in many other cell types and storage products are present in body fluids. The level of CTH in tissues, plasma or urine can be used to follow the course of the disease or conversely to monitor treatment.

Several methods have been used to measure CTH directly, including TLC (Berna L et al., (1999) Anal. Biochem. 269,304-311, TLC with immunodetection (Yiu S C, Lingwood Calif. (1992) Anal. Biochem.202, 188-192), TLC with immunoblotting of the glycolipids onto nitrocellulose (Towbin H et al., (1984) J.Immunol.Methods 72, 471-479) or polyvinylidene difluoride (Taki T et al., (1994) Anal. Biochem. 221, 312-316) membranes.

Alternatively the oligosaccharide moiety can be released and measured by GLC (Vance D E, Sweeley C C. (1967) J. Lipid. Res. 8, 621-630) or HPLC (McCluer R H et al., (1989) Methods Enzymol. 172, 538-575; Schiffman R et al., (2000) Proc. Natl. Acad.Sci.USA 97, 365-370). An ELISA method using Verotoxin subunit B, which obviates many of the time consuming steps in the other methods, has also been developed (Zeidner K M et al., (1999) Analytical Biochemistry 267, 104-113). Various forms of mass spectrometry have been used for the direct detection and characterisation of the glycosphingolipids in biological samples (Domon B, Costello CE (1988) Biochemistry 27, 1534-1543; Ii et al., 1995; Guittard et al., (1999) Rapid Commun. Mass Spectrom. 13, 1838-1849; Hsu and Turk (2001) J. Am. Soc. Mass Spectrom. 12, 61-79).

The advantages of mass spectrometry over the other methods are specificity, sensitivity rapid throughput and potential for automation. The main drawback with existing mass spectrometry methods for measuring sphingolipids is the lack of internal standards, which are necessary for quantitation of the method. Internal standards are compounds that are chemically identical or very similar to the compound to be measured but have a different mass so that they can be detected in the presence of the test compound by mass spectrometry. A known amount of the internal standard is added to the biological sample to be analysed right at the beginning of the analysis. As it is taken through the same procedure as the compound to be assayed, its recovery should be the same. Comparison of the analytical responses of a known amount of the standard and unknown amount of the test compound in a biological sample enables the concentration of the latter to be measured accurately.

An appropriate internal standard should resemble the test compound as closely as possible and have the same function and configuration as the test compound. There are two types of internal standard: type 1 is identical chemically to the compound to be measured but differs in mass by virtue of incorporation of radioactive or non-radioactive isotopes e.g. ¹³C for carbon or deuterium for hydrogen; type 2 differs slightly in structure and hence has a different mass but is otherwise chemically as similar as possible to the test compound.

Internal standards for many biological compounds have been developed, for example U.S. Pat. No. 6,258,605 describes isotope internal standards for acyl carnitines and alpha amino acids and International patent specification WO/014747 discloses isotopically labelled standards comprising ¹³C and ²H used in tandem mass spectrometry to screen for metabolic disorders of fatty acid metabolism. However, there are no internal standards available for more complex compounds such as sphingolipids, possibly because of the complex structure of these compounds.

It is an aim of a preferred embodiment of the present invention to obviate or mitigate a problem associated with the prior art, whether referred to herein or otherwise, and to provide internal standards for sphingolipids which allow measurement of sphingolipids by mass spectrometry.

Accordingly the present invention provides an internal standard for use in mass spectrometry to determine the concentration of a test sphingolipid, the internal standard comprising the same oligosaccharyl chain and long chain base sphingosine as the test sphingolipid and having an acyl group of different mass from the test sphingolipid but providing a sphingolipid of the same chemical nature as the test sphingolipid.

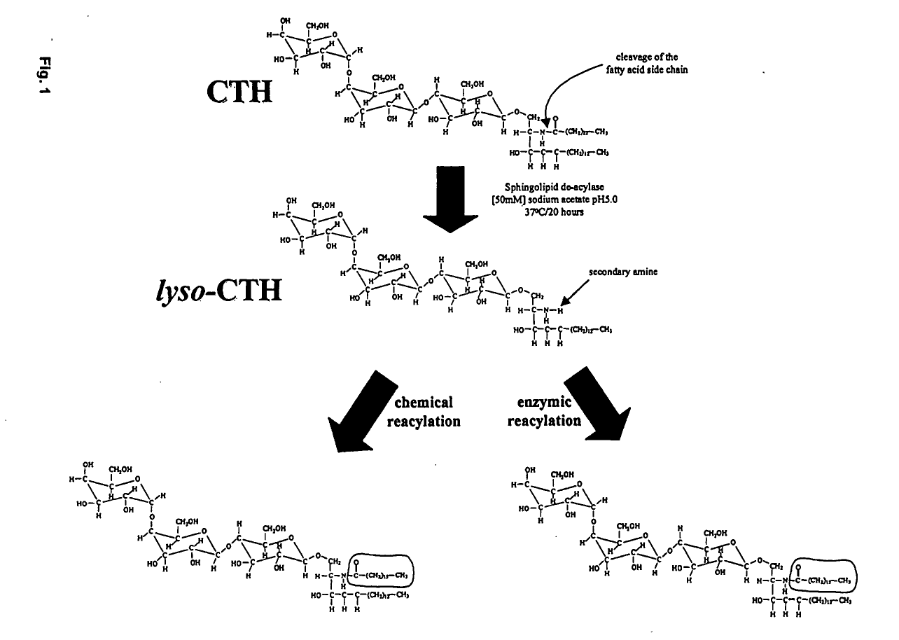

CTH consists of three components as shown in FIG. 1, an oligosaccharyl chain, a long chain base (LCB) sphingosine and a fatty acid (acyl group). When designing internal standards for CTH the inventors found that the oligosaccharide chain and long chain base are characteristic of CTH and cannot be changed without altering the chemical nature of CTH. They found that the acyl chain, which is heterogeneous in naturally occurring CTH, can be substituted to include a mass marker to produce type 1 and type 2 internal standards.

A preferred embodiment of the invention provides a type 1 internal standard for a sphingolipid in which the acyl group has the same number of carbon atoms as the test sphingolipid but comprises an isotopic label.

Preferably, the isotopic label comprises a hydrogen isotope, especially deuterium. The isotopic label may also comprise ¹³C.

Preferably, the invention provides sphingolipids with deuterated acyl groups, which are chemically identical to natural sphingolipids but differ in mass, allowing them to be detected by mass spectrometry.

According to a preferred embodiment the present invention provides [D4] C-16 CTH, which is chemically identical to the predominant C-16 CTH in blood and is a type 1 internal standard.

In nature compounds containing acyl groups generally have acyl groups with an even number of carbon atoms. A preferred embodiment of the invention provides a type 2 internal standard for a sphingolipid in which the acyl group comprises an odd number of carbon atoms. Preferably, the acyl group comprises one more or one less carbon atom than the test sphingolipid. These acyl groups with an odd number of carbon atoms do not occur naturally but are chemically very similar to the test sphingolipid, differing in mass by factors of 14, preferably by only 14.

For example, human blood CTH normally contains a mixture of acyl groups with an even number of carbon atoms but the C-16 acyl group predominates. The invention provides C17-CTH, which has an acyl group of 17 carbon atoms. C-17 CTH does not occur naturally but is chemically very similar to the natural C-16 acyl group and differs in mass by 14 and is a type 2 internal standard.

The invention also provides, according to preferred embodiments, C-15, C-19, C21, C-23 and C-25 acyl sphingolipids.

Specifically, the invention provides C-15 CTH, C-17 CTH, C-19 CTH, C-21 CTH, C-23 CTH and C-25 CTH. In a second aspect the present invention provides methods for preparation of internal standards for sphingolipids.

To make both type 1 and type 2 internal standards an acyl group carrying a label that is detectable by mass spectrometry is added to the lyso-form of the test sphingolipid.

The lyso-form of a sphingolipid is the N-deacylated form, which lacks the fatty acid acyl group which is normally attached to the amino group of the sphingoid through a acid amide bond.

Some lyso-sphingolipids are commercially available, such as lyso-CTH which is produced by Calbiochem, Calif., USA. As other lyso-sphingolipids are not commercially available or are very expensive the inventors use sphingolipid ceramide N-deacylase to produce lyso-sphingolipids (see European patent specification EP 1 067 196 A1).

According to a first embodiment of the second aspect of the invention the mass marker may be added to the lyso-sphingolipid chemically.

The chemical synthesis of a type 1 internal standard from a lyso-sphingolipid comprises the addition of a deuterated even numbered chain fatty acyl group to the lyso-sphingolipid in the presence of organic solvents and anhydrides.

The chemical synthesis of a type 2 internal standard from a lyso-sphingolipid comprises the addition of an odd numbered chain fatty acyl group to the lyso-sphingolipid in the presence of organic solvents and anhydrides.

The examples show the chemical preparation of C-17 CTH from lyso-sphingolipid. This process is equally applicable to other acyl groups and other sphingolipids.

According to a second embodiment of the second aspect of the invention the mass marker may be added to the lyso-sphingolipid enzymically by exploiting the reverse reaction of sphingolipid ceramide N-deacylase under specific conditions (see European patent specification EP 0 940 409). The examples show the enzymic preparation of C-17 CTH and [D4]C16-CTH from lyso-sphingolipid using sphingolipid ceramide N-deacylase. This process is equally applicable to other acyl groups and other sphingolipids.

The present invention in a third aspect provides the use of the internal standards according to the first aspect of the invention or made according to the method of the second aspect of the invention in mass spectrometry.

Preferably the internal standards are used in electrospray ionisation-tandem mass spectrometry (ESI-MS).

Electrospray tandem mass spectrometry is very sensitive and specific and can detect a broad spectrum of disorders

The present invention in a fourth aspect provides a method of diagnosis or monitoring therapy of lysosomal storage diseases comprising analysing a biological sample for levels of sphingolipid by mass spectrometry using an internal standard according to the first aspect of the invention or made according to the method of the second aspect of the invention.

Preferably the biological sample comprises a blood, plasma, tissue or urine sample.

Newborn infants are screened for metabolic disorders in all developed countries of the world and is beginning to be adopted by developing countries. Prior to the use of tandem mass spectrometry the number of disorders screened for has been limited in most programmes to less than six.

To increase the number of disorders screened for tandem mass spectrometry has been adopted. This allows screening for multiple disorders from a single blood spot or small volume of plasma or urine.

The standard method of collecting samples for neonatal screening is a heel prick followed by depositing the whole blood on special filter paper or Guthrie cards as a series of spots (see Millington, et al., Int. J. Mass Spectrometry and Ion Processes, 111, 212 1991). More recent methods consist of processing samples in microplates also containing internal standards.

The present invention provides an additional set of internal standards which can be used in tandem mass spectrometry of heel prick tests from newborns to allow for diagnosis of lysosomal storage diseases. The internal standards can also be used on samples from adults for diagnosis of the diseases or to monitor the effect of treatment.

The present invention will now be described, by way of example only, with reference to the following drawings, in which:

FIG. 1 shows methods for synthesis of CTH internal standards according to a second aspect of the invention;

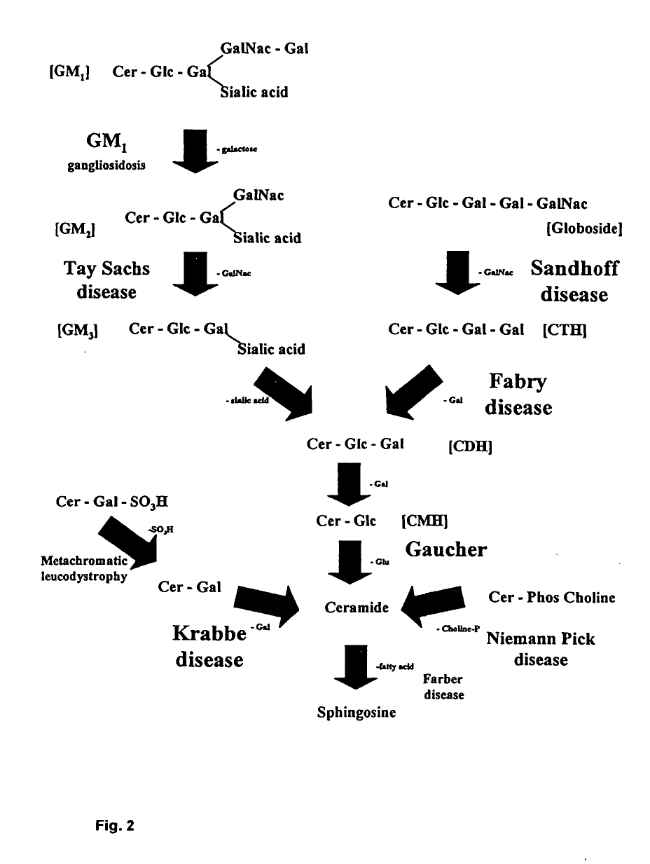

FIG. 2 shows the pathway for lysosomal catabolism of sphingolipids;

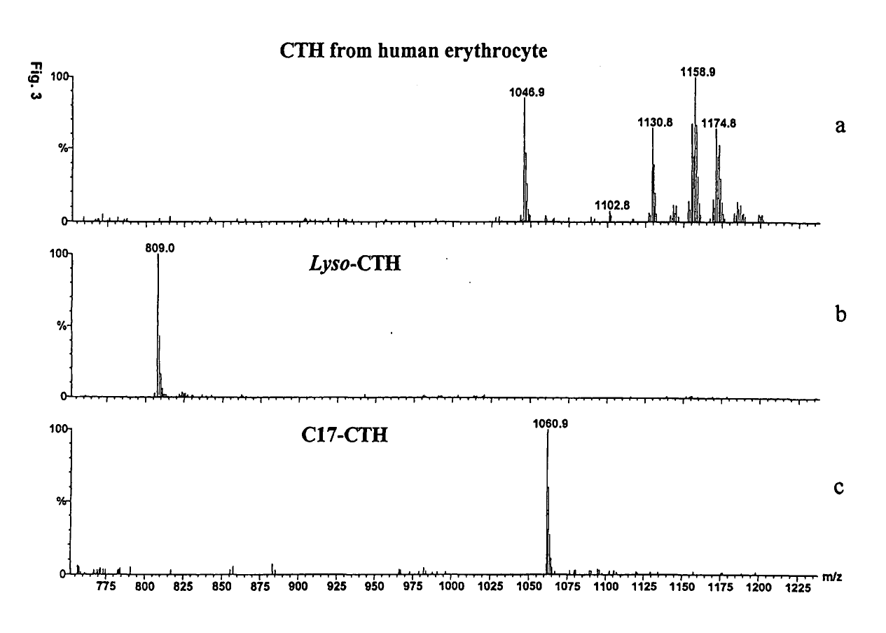

FIG. 3 shows mass spectrometry of the synthesis of C-17 CTH;

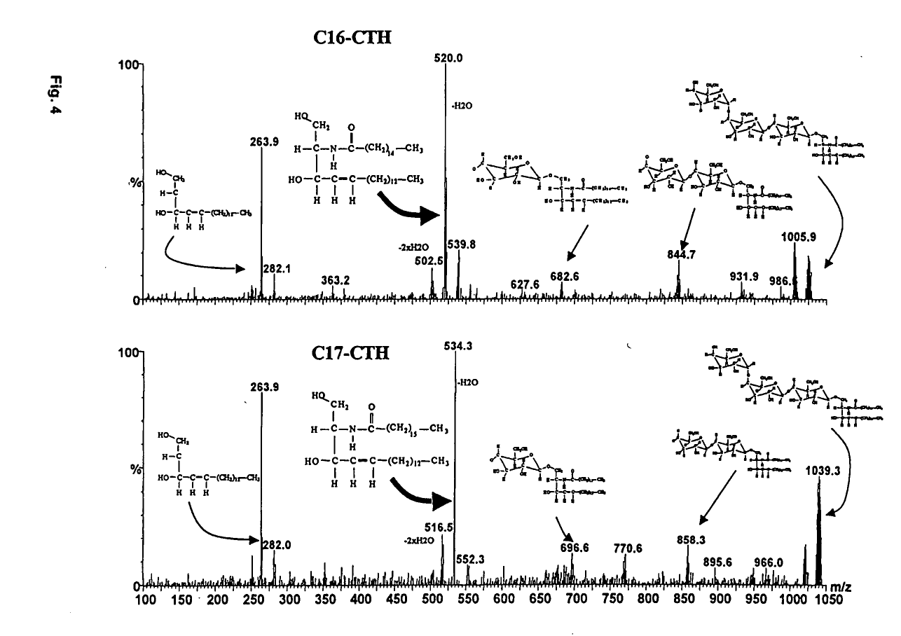

FIG. 4 shows an ESI/MS spectrum confirming the structure of the product of acylation of lyso-CTH;

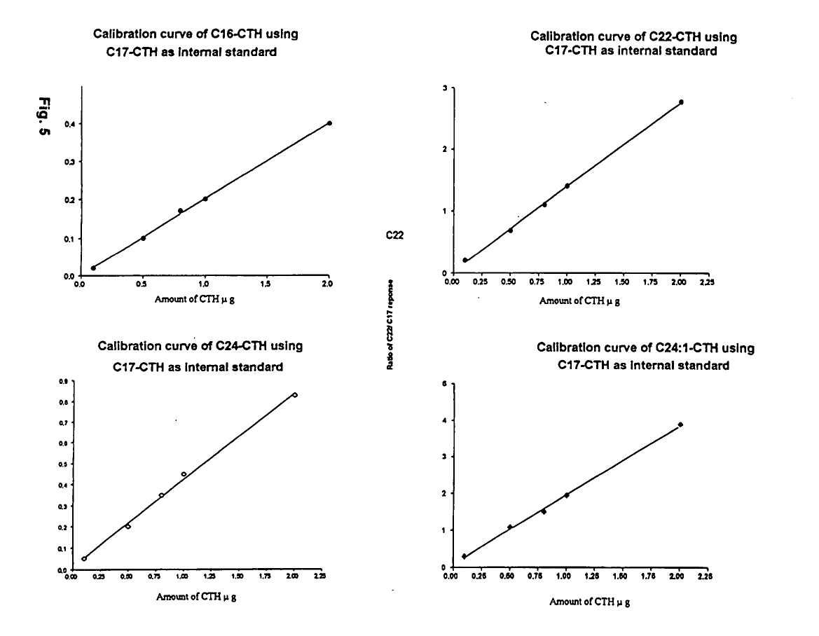

FIG. 5 shows the calibration of C-17 CTH;

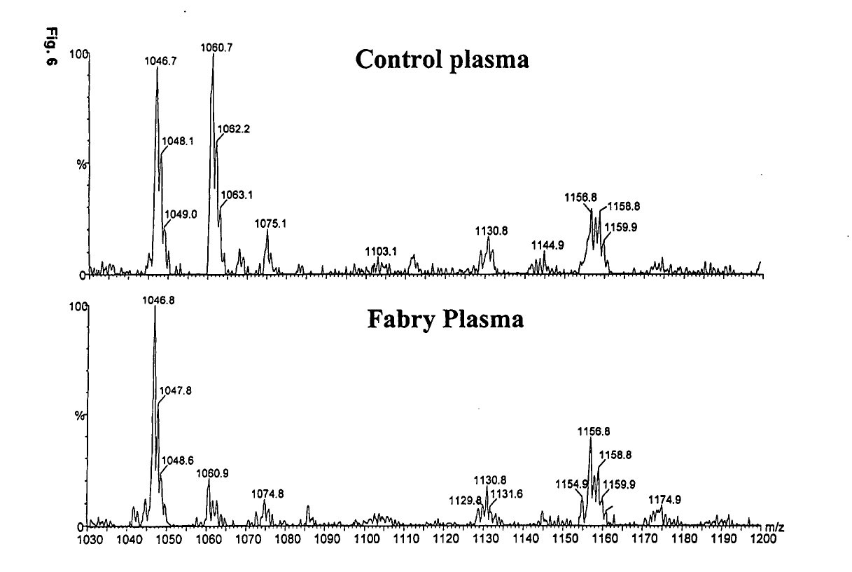

FIG. 6 shows an analysis of CTH in normal control plasma and plasma from patients with classic Fabry disease; and

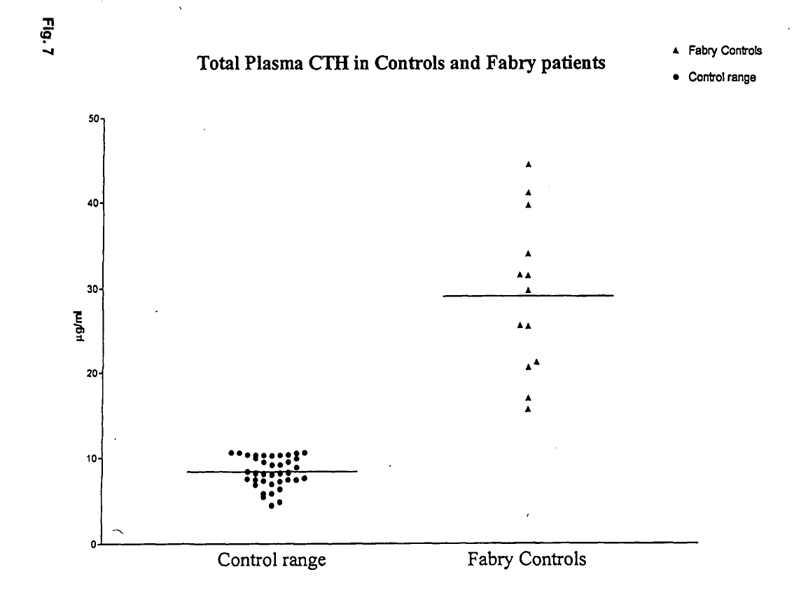

FIG. 7 shows the difference between total plasma CTH levels in control and Fabry patients as determined using mass spectrometry with the internal standards according to the present invention.

### EXAMPLES

The present invention is illustrated below by the synthesis and use of CTH for the monitoring of Fabry disease but in principle can be applied to the whole pathway for the lysosmal catabolism of glycosphingolipids (see FIG. 2).

Materials

Plasma and urine were obtained from male patients with the classic form of Fabry disease and from normal controls with informed consent.

Human erythrocyte CTH, (Galα(1→4)Galα(1→4)Glc-ceramide (globotriaosylceramide, GbOse3Cer, Gb₃, GL-3 or CTH) was obtained from Sigma (Poole, Dorset, UK).

Sphingolipid ceramide N-deacylase and lyso-CTH (Gala(1→4)Galp(1→4)Glc-sphingosine) were obtained from Calbiochem, Calif., USA.

All chemical reagents were of Analar grade from Sigma.

Chemical Synthesis of C-17 CTH.

50 kg of lyso-CTH was dried completely under N₂ in a glass vial and then dissolved in 100 μl of heptadecanoic acid anhydride in chloroform (10 mg/ml). Pyridine (300 μl) was added and the solution was vortexed thoroughly and left at room temperature for 30 min. After drying completely under nitrogen to remove all the excess pyridine, 100 μl of heptadecanoic acid anhydride in chloroform (100 mg/ml) and 300 μl of pyridine were added to the reaction mixture, which was again thoroughly mixed and left at room temperature for 30 min. The reaction mixture was dried under nitrogen and desalted using a C-18 solid phase extraction cartridge (LiChrolute, Merck, Poole, Dorset, UK) as described by Zeidner et al., (1999) supra.

Enzymic Synthesis of C-17 CTH and [D4]C16-CTH

50 μg (˜50 nmol) of human erythrocyte CTH was incubated in 20 μl of sodium acetate buffer, pH 5.0 with 1 mUnit of sphingolipid ceramide N-deacylase for 16 h at 37° C. essentially as described by Ito et al., (1995) except that Triton X-100 was replaced by fatty acid-free 1% (w/v) BSA. The reaction mixture was freeze-dried and the lipid products were extracted in 5 μl of chloroform/methanol (1:2, v/v). The extent of the enzymatic deacylation was determined by TLC on silica gel plates (Silica Gel 60, Merck) using chloroform: methanol:acetic acid (10%) (5:4:1) as the mobile phase. The glycosphingolipids and lyso-glycosphingolipids were detected using iodine vapour and identified by comparison with standards. Lyso-CTH was scraped off the TLC plate and recovered from the silica using two washes of 500 μl of chloroform:methanol (2:1, v/v).

In the second enzymic step, purified or commercial lyso-CTH (75 nmol) was incubated with 75 nmol of heptadecanoic acid or [D4]-palmitic acid and 200 μU of sphingolipid ceramide N-deacylase in 20 μl of 25 mM sodium phosphate buffer, pH, 6.5, (containing fatty acid-free 1% BSA (w/v)), for 20 h at 3720 C. The products of the re-acylation reaction were analysed using TLC as described above with >90% conversion of lyso-CTH to CTH observed.

For larger-scale preparation of the internal standards, the reaction products were desalted using a C-18 solid-phase extraction cartridge (LiChrolute, Merck). The C18 cartridge was first primed with 5 ml of methanol, followed by 5 ml of H₂O. The reaction mixture was added directly to the cartridge and the salts and residual detergent present in the enzyme preparation, were removed by successive washing with 3 ml H₂O, 3 ml of 60% methanol and 3 ml of 70% methanol. The CTH internal standards were eluted from the column using 2 ml of methanol followed by 2 ml chloroform:methanol (2:1 v/v).

Extraction of CTH from Plasma and Preparation for Analysis

1 μg of CTH internal standard was added to 100 μl of plasma, from which the total lipid was extracted by the addition of 2 ml of chloroform:methanol (2:1 v/v) with shaking on a multivortexer for 20 min. Any precipitated protein was removed by centrifugation at 3000 g for 10 min. The chloroform: methanol (2:1 v/v) layer was transferred to a vial and 400 μl of H₂O was added and the vial was shaken for a further 20 min. The two layers were separated by centrifugation for 10 min and the lower layer containing the neutral glycolipids including the CTH was transferred to a new vial and dried under N₂. The CTH was desalted prior to mass spectrometry by reconstitution in chloroform followed by chromatography on a C-18 column (LiChrolute) and elution with acetone:methanol (9:1) according to Zeidner et al. (1999) supra.

Electrospray Ionisation-Tandem Mass Spectrometry (ESI-MS):

Mass spectrometry of glycolipids was carried out using a triple quadropole VG Quattro I instrument (MicroMass, Altrincham, U.K.). The instrument was operated in positive ionisation mode. Samples were directly infused into the electrospray source via a 25 mm (i.d.) fused silica transfer line by means of a Harvard syringe pump at a flow rate of 25 μl/min. The capillary voltage was maintained at 3.43 kV with a cone voltage 173 V. The source temperature was held constant at 80° C. and nitrogen was used as the nebulising gas at a flow rate 30 litres/hour. The masses of all the CTH isoforms were determined by operating the mass spectrometer in scan mode over a mass range of m/z 700-1300. Product ions were determined over a mass range of m/z 50-1200 following collision-induced dissociation using argon as the collision gas. The optimum collision energy was determined to be 70 eV with an optimum gas cell pressure of 3.2×10⁻³ mbar. Data were acquired using neutral loss scanning of m/z 162.0, operating in multiple channel acquisition mode and with a dwell time for each ion species of 100 msec. For quantitative analyses samples were infused into the mass spectrometer using the electrospray ion source and CMA/200 refrigerated auto sampler.

Liquid Secondary Ionisation-Tandem Mass Spectrometry (LSI-MS):

To confirm the structures of the individual CTH isoforms and the internal standard, the CTH isoforms were analysed using the tandem mass spectrometer operating with a liquid secondary ionisation source (LSIMS). This ionisation mode produced more extensive fragmentation through the ceramide backbone of the CTH, which could not be achieved using the ESI source, and allowed definitive determination of the acyl group in each CTH isoform, Mass spectrometry of glycolipids was carried out using a triple quadropole VG Quattro I instrument (MicroMass, Altrincham, U.K.). The instrument was fitted with a LSIMS source (CsI) operating with an accelerating voltage of 9 kV in positive ionisation mode. MS/MS experiments were conducted using a collision energy of 30 eV and a gas cell pressure of 3.2×10⁻³ mBar (argon).

Results

Chemical and Enzymic Synthesis of C17-CTH

Human erythrocyte CTH consists of a mixture of isoforms, which differ in the length of the acyl chain (FIG. 3a). The most abundant forms contain lignoceric acid (C24:0) and nervonic acid (C24:1) with appreciable amounts of their hydroxylated derivatives. Other constituents are the C24:2 acid, behenic acid (C22:0) and its monounsaturated derivative (C22:1) and palmitic acid (C16:0) isoforms. All of the ions in the spectrum could be attributed to mono-sodiated CTH isoforms, indicating that the sample was very pure and could be used to calibrate novel standards. The masses of the ions suggest that the sphingosine moiety is d-18:1. The structure of the sphingosine moiety was confirmed by LSI-MS mass spectrometry, which showed a characteristic fragmentation ion of 264 m/z/ corresponding to sphingosine d-18:1 minus H₂O.

The ESI/MS spectrum for lyso-CTH consisted predominantly of an ion of 809.0 m/z, which corresponds to a mono-sodiated Galα(→4)Galβ(1→4)Glc-sphingosine (d-18) (FIG. 3b). The ESI/MS spectrum for the product of the acylation of lyso-CTH had a molecular ion of 1060.9 m/z, consistent with the formation C-17 CTH (CTH d18:1 C17-1H₂O) (FIG. 3c). The structure of the C17-CTH was confirmed by LSI-MS (FIG. 4). The fragmentation pattern contained ions of 1039.3 m/z, 534.3 m/z and 264.0 m/z corresponding to the molecular ion and the daughter ions for the ceramide Cer(d18:1 C17-2H₂O) and sphingosine (d18:1) moieties, respectively.

As the yield for the chemical synthesis of C-17CTH was very low (1-10%), the specificity of enzymes was exploited to make C-17CTH. Firstly, lyso-CTH was prepared from the commercially available erythrocyte CTH by using the hydrolytic activity of sphingolipid ceramide N-deacylase (Ito et al., (1995) J. Biol. Chem. Vol. 270, No. 41, 24370-24374). Lyso-CTH was purified from the reaction mixture by TLC and its structure established by a combination of ESI-MS/MS and LSI-MS/MS. C17-CTH was synthesised enzymically from lyso-CTH, which had been prepared enzymically or commercially, using the reverse reaction of sphingolipid ceramide N-deacylase as described by Mitsutake. et al., (1998) Anal. Biochem. 247, 52-57). The C17-CTH was purified from the enzymic reaction mixture as described above and analysed by ESI/MS. The spectrum was identical to that obtained for the chemically synthesised compound (FIG. 3c) and its structure was confirmed by LSI-MS as for the chemically synthesised molecule. The yield for the enzymic acylation reaction was >90% as determined by the ratio of the lyso-CTH to CTH after TLC. Therefore it was possible to make the C17-CTH from human CTH by a two-step enzymic process or directly from commercially available lyso-CTH in very high yields in excess of >90%.

Measurement of CTH in Plasma Using C-17CTH Internal Standard

The C-17 CTH standard was calibrated by mixing a constant amount of the standard with different amounts of the authentic CTH and analysing the mixtures by ESI/MS. (FIG. 5). A linear relationship between the ratio of responses and the amount of authentic CTH was observed for each isoform over an appropriate range. This enabled the C-17 CTH to be calibrated in terms of weight of authentic CTH isomer. To use the C17-CTH standard to measure the concentration of CTH in plasma, 1 μg of the standard was added to 100 μl of plasma and the total CTH was extracted and analysed by ESI/MS. Replicate analysis (12 times) of the CTH in a single normal plasma sample gave a coefficient of variation of 9.9%.

This procedure was used to analyse the CTH in normal control plasma and plasma from male patients with classic Fabry disease (FIG. 6). It can be seen from FIG. 7 that the levels of all the forms of CTH are elevated in the plasma from the patients with Fabry disease. The level of C-16 CTH, which accounted for ˜50% of the total CTH, was particularly increased, indicating that it is the major storage product in plasma in this group of patients. The levels of the total CTH and of the individual isoforms were measured quantitatively by neutral loss scanning of m/z 162.0, operating in multiple channel acquisition mode (FIG. 5). The mean level of total CTH in the normal plasma was 8.4 μg/ml, (range 4.5-10.7, n=38) and in the Fabry plasma, 29.1 μg/ml (range 15.8-44.5, n=13).

Enzymic Synthesis of [D4]C16-CTH, a Type 1 Internal Standard for Measurement of C-16CTH

[D4]C-16CTH was also synthesised enzymically by substituting [D4]palmitic acid for the heptadecanoic in the second step of the enzymic procedure. The structure of the [D4]C-16CTH was confirmed by a combination of ESI-MS and LSI-MS as for the C-17CTH. Replicate analysis (11 times) of the CTH in a single normal plasma sample using the [D4]C-16CTH as an internal standard gave a coefficient of variation of 7.9% and values within the normal control reference range determined using C-17 CTH as an internal standard.

Our values for total plasma CTH in normal or Fabry samples are slightly higher than those found by GLC (Vance et al., 1969 supra) or by an ELISA method using verotoxin B, 0.9 μg/ml (range 0.3-1.5).

1. A method for the determination of the concentration of a test glycosphingolipid by mass spectrometry comprising the provision of an internal standard glycosphingolipid, the internal standard comprising the same oligosaccharyl chain and long chain base glycosphingosine as the test glycosphingolipid and having an acyl group of different mass from the test glycosphingolipid but providing a glycosphingolipid of the same chemical nature as the test glycosphingolipid:

2. The method of claim 1 in which the internal standard is of type 1 in which the acyl group has the same number of carbon atoms as the test glycosphingolipid and further comprises an isotopic label.

3. The method of claim 2 in which the isotopic label comprises a hydrogen isotope or carbon isotope.

4. The method of claim 3 in which the glycosphingolipid comprises deuterated or ¹³C acyl groups, wherein said glycosphingolipid is chemically identical to natural glycosphingolipids but differs in mass, allowing it to be detected by mass spectrometry.

5. The method of claim 4 comprising [D4] C-16 ceramide trihexoside (CTH).

6. The method of claim 1 in which the internal standard is of type 2 in which the acyl group of the glycosphingolipid comprises an odd number of carbon atoms.

7. The method of claim 6 in which the acyl group of the internal standard comprises one more or one less carbon atom than the acyl group of the test glycosphingolipid.

8. The method of claim 6 comprising one or more of C-15, C-17, C-19, C-21, C-23 and C-25 acyl glycosphingolipids.

9. The method of claim 6 comprising one or more of C-15 CTH, C-17 CTH, C-19 CTH, C-21 CTH, C-23 CTH and C-25 CTH.

10. The method of claim 1 wherein the mass spectrometry is electrospray ionization-tandem mass spectrometry.

11. A method for diagnosing lysosomal storage diseases comprising analyzing a biological sample for levels of glycosphingolipid by mass spectrometry using an internal standard in accordance with claim 1.

12. The method according to claim 11 in which the biological sample comprises a sample of blood, plasma, tissue or urine.

13. A method according to claim 12 in which the sample comprises a heel prick sample from an infant.

14. An internal standard for use in mass spectrometry in the determination of the concentration of a test glycosphingolipid, the internal standard comprising the same oligosaccharyl chain and long chain base glycosphingosine as the test glycosphingolipid and having an acyl group of different mass from the test glycosphingolipid but providing a glycosphingolipid of the same chemical nature as the test glycosphingolipid.

15. A method for preparation of an internal standard for test glycosphingolipids as claimed in claim 14 comprising adding an acyl group carrying a label that is detectable by mass spectrometry to the lyso form of the test glycosphingolipid.

16. The method of claim 15 in which the label is a mass marker is added to the lyso-glycosphingolipid chemically.

17. The method of claim 15 in which the label is a mass marker added to the lyso-glycosphingolipid enzymically by using the reverse reaction of glycoshpingolipid ceramide N-deacylase.

18. The method according to claim 11 wherein the mass spectrometry is electrospray ionization-tandem mass spectrometry.

19. The internal standard according to claim 14, wherein the mass spectrometry is electrospray ionization-tandem mass spectrometry.
